(19) United States
(12) Patent Application Publication
(10) Pub. No.: US 2025/0210146 A1
(43) Pub. Date: Jun. 26, 2025

(54) SEQUENCE OPTIMIZATION

(71) Applicant: UAB Biomatter Designs, Vilnius (LT)

(72) Inventors: Donatus Repecka, Vilnius (LT); Irmantas Rokaitis, Vilnius (LT); Vykintas Jauniškis, Vilnius (LT); Diana Ikasalaite, Vilnius (LT); Laurynas Karpus, Vilnius (LT)

(21) Appl. No.: 18/847,448

(22) PCT Filed: Mar. 16, 2023
(86) PCT No.: PCT/EP2023/056787

§ 371 Date: Sep. 16, 2024

(30) Foreign Application Priority Data
Mar. 17, 2022 (GB) 2203714.7

(51) Int. Cl. G16B 40/30; G16B 20/50

(52) U.S. Cl. CPC G16B 40/30; G16B 20/50

(57) ABSTRACT

Provided herein is an apparatus for generating an optimized protein or nucleic acid sequence from a target protein or nucleic acid sequence, wherein the optimized protein or nucleic acid sequence has an improved function over the target sequence. The apparatus comprises at least one processor and at least one memory including a computer program code, the at least one memory and computer program code configured to, with the at least one processor, cause the apparatus to at least: operate a machine learning model configured to receive the target protein sequence or a nucleic acid sequence, and to generate therefrom one or more corresponding optimized sequences, wherein the machine learning model has been trained on a set of training data comprising native or engineered protein or nucleic acid sequences, and additionally, at least a subset of the sequences comprising one or more masked portions and/or at least a subset of the sequences comprising one or more mutations introduced therein.

### FIELD OF THE INVENTION

The present invention generally relates to machine learning-guided protein and nucleic sequence optimization. More specifically, the present invention relates to an apparatus for generating an optimized protein or nucleic acid sequence from a target protein or nucleic acid sequence, and a computer-implemented method for generating the same.

### BACKGROUND OF THE INVENTION

Nature has provided countless proteins with significant yet often unexplored potential for technological, scientific and medical applications. However, proteins in their natural or native forms rarely function optimally for their envisioned uses. Thus, proteins often benefit from sequence engineering to enhance their functionality and performance.

One way of engineering proteins is through directed evolution. Direct evolution mimics the process of natural selection to steer proteins or nucleic acids towards a user-defined goal or “fitness” level. Directed evolution is a laboratory-based process which involves introducing mutations into existing proteins (which may be sourced from nature or engineered), screening for progeny proteins with enhanced activity (or another desirable trait) and selecting those with a desired level of performance. The process is then repeated in an iterative fashion using the selected proteins until a target level of performance is achieved. However, such directed evolution techniques can result in the generation of thousands of variants with each round of mutagenesis, and implementing a suitable screen or selection can represent a significant experimental burden in terms of both cost and time. There is therefore the need to provide methods of generating and screening protein variants in silico.

Molecular dynamics simulations which predict dynamic structural changes for protein variants, have been used to predict changes in structure and protein properties caused by mutations. However, full simulations are also resource-intensive, requiring hundreds of processor hours for each variant, a mechanistic understanding of the reaction at hand, and, ideally, a reference protein structure which may not always be available.

### SUMMARY OF THE INVENTION

In order to overcome the constraints imposed by directed evolution and other protein engineering techniques, the invention provides machine learning-guided protein and nucleic acid optimization based on a target protein or nucleic acid sequence.

Accordingly, in a first aspect, an apparatus is provided for generating an optimized protein or nucleic acid sequence from a target protein or nucleic acid sequence, the apparatus comprising:
at least one processor and at least one memory including a computer program code, the at least one memory and computer program code configured to, with the at least one processor, cause the apparatus to at least:
operate a machine learning model configured to receive an input target protein sequence or a nucleic acid sequence, and to generate therefrom one or more corresponding optimized sequences having an improved function over the target sequence, each optimized sequence having one or more mutations with respect to the target sequence, wherein the machine learning model has been trained on a set of training data comprising native or engineered protein or nucleic acid sequences, and additionally, at least a subset of the sequences comprising one or more masked portions and/or at least a subset of the sequences comprising one or mutations introduced therein; and
generate output data relating to the one or more optimized sequences.

Preferably, the apparatus is configured to perform the following steps after the target sequence has been received:
i) determine the likelihoods of substitutions with a predefined set of proteinogenic amino acids at one or more positions within the target sequence when the target sequence is a protein sequence, or with a predefined set of bases at one or more positions within the target sequence when the target sequence is a nucleic acid sequence;
ii) calculate scores for the likelihoods of the substitutions at the one or more positions based on a scoring function;
iii) select one or more substitutions based on calculated scores to generate one or more new mutated target sequences each comprising one or more substitutions;
iv) repeat steps i) to iii) until no further substitutions with an improved score are yielded at each corresponding position; or repeat steps i) to iii) until the apparatus has performed for a selected amount of time.

In a preferred embodiment, in step i), the apparatus is configured to determine the likelihoods of substitutions with a pre-defined set of proteinogenic amino acids at two or more respective positions within the target sequence when the target sequence is a protein sequence, or with a pre-defined set of bases at two or more respective positions within the target sequence when the target sequence is a nucleic acid sequence, wherein the likelihoods are based on the combined substitutions at the two or more positions.

In a second aspect, the present invention provides a computer-implemented method of generating one or more optimized protein sequences or nucleic acid sequences from a target protein sequence or nucleic acid sequence, using a trained machine learning model. In the method, the machine learning model has been trained based on:
a) a set of training data comprising native or engineered protein or nucleic acid sequences; and
b) at least a subset of the native or engineered protein or nucleic acid sequences comprising a masked portion and/or at least a subset of the native or engineered protein or nucleic acid sequences comprising one or mutations introduced therein.

The machine learning model is configured to use the training data in order to become a trained machine learning model.

The method comprises:
i) receiving as input the target sequence;
ii) causing the trained machine learning model to evaluate the inputted target sequence; and
iii) based on the evaluation, generating one or more optimized sequences corresponding to the target sequence, each optimized sequence having an improved function over the target sequence, and one or more mutations with respect to the target sequence; and
iv) outputting data relating to the one or more generated optimized sequences.

Preferably, in step (ii), evaluation of the target sequence comprises:
v) determining the likelihoods of substitutions with a pre-defined set of proteinogenic amino acids at one or more positions within the target sequence when the target sequence is a protein sequence, or with a pre-defined set of bases at one or more positions within the target sequence when the target sequence is a nucleic acid sequence;
vi) calculating scores for the likelihoods of the substitutions at the one or more positions based on a scoring function;
vii) selecting one or more substitutions based on calculated scores to generate one or more new mutated target sequences each comprising one or more substitutions;
viii) repeating steps v) to vii) until no further substitutions with an improved score are yielded at each corresponding position; or repeating steps v) to vii) until the apparatus has performed for a selected amount of time.

Other preferred features of the invention are set out in the appended dependent claims.

### BRIEF DESCRIPTION OF THE FIGURES

Reference is made to the following description and drawings, in which:

FIG. 1 is a schematic representation of an apparatus according to the present invention.

FIG. 2 is a flow chart illustrating the optimization of a target sequence.

FIG. 3 is an SDS-PAGE gel illustrating expression of optimised variants of beta-glucosidase (OP5-OP8).

FIG. 4 is an SDS-PAGE gel illustrating purified optimised variants of beta-glucosidase (OP5-OP8).

FIG. 5 is a bar chart illustrating the activity of optimised variants of beta-glucosidase (OP5-OP8).

FIG. 6 is a bar chart illustrating Tm values of optimised variants of beta-glucosidase (OP5-OP8).

FIG. 7A is a line graph illustrating residual activity of optimised variants of beta-glucosidase (OP5-OP8) after heating to selected temperatures for 5 minutes.

FIG. 7B is a line graph illustrating residual activity of optimised variants of beta-glucosidase (OP5-OP8) after heating to selected temperatures for 30 minutes.

FIG. 7C is a line graph illustrating residual activity of optimised variants of beta-glucosidase (OP5-OP8) after heating to selected temperatures for 60 minutes.

FIG. 8 is a bar chart illustrating the activity of optimised variants of beta-glucosidase (OP9-OP11).

FIG. 9 is a bar chart illustrating Tm values of optimised variants of beta-glucosidase (OP9-OP11).

FIG. 10 is a bar chart illustrating the activity of optimised variants of beta-glucosidase (OP12-OP18).

FIG. 11 is a bar chart illustrating Tm values of optimised variants of beta-glucosidase (OP12-OP18).

FIG. 12 is an SDS-PAGE gel illustrating expression of optimised variants of beta-glucosidase (OP1-OP4).

FIG. 13 is an SDS-PAGE gel illustrating purified optimised variants of beta-glucosidase (OP1-OP4).

FIG. 14 is a bar chart illustrating the activity of optimised variants of flavin reductase hypE (OP1-OP4).

FIG. 15 is a bar chart illustrating Tm values of optimised variants of flavin reductase hypE (OP1-OP4).

FIG. 16 is a line graph illustrating residual activity of optimised variants of flavin reductase hypE (OP1-OP4) after heating to selected temperatures for 60 minutes.

### DETAILED DESCRIPTION

In the following description and examples, improved means for generating optimized protein or nucleic acid sequences are disclosed. Machine learning-based methods may be used for screening protein function in silico. Use of machine learning essentially comprises a set of algorithms that make decisions based on data. Machine learning approaches involve the prediction of relationships between sequences and function in a data-driven manner without requiring a detailed model of underlying physical or biological pathways. Such methods accelerate directed evolution by learning from the properties of characterized variants and using that information to select sequences that are likely to exhibit improved properties. Furthermore, while conventional directed evolution discards information from unimproved sequences (i.e. only those with improved properties are selected and are subjected to further mutagenesis), machine-learning based methods can use this information to expedite evolution and expand the properties that can be optimized by intelligently selecting new variants to screen, reaching higher fitness levels than through directed evolution alone.

A benefit of machine learning is in reducing the quantity of sequences to test experimentally. Therefore, machine learning is particularly useful in cases where a lack of a high-throughput screen limits or precludes directed evolution. The following description and examples illustrate how extended periods of search time to identify likely optimized sequences can be avoided, and how machine learning methods can be used to generate optimized sequences efficiently.

In one aspect, an apparatus for generating an optimized protein or nucleic acid sequence from a target protein or nucleic acid sequence is provided, the apparatus comprising at least one processor and at least one memory including a computer program code, the at least one memory and computer program code configured to, with the at least one processor, cause the apparatus to at least:

operate a machine learning model configured to receive an input target protein sequence or a nucleic acid sequence, and to generate therefrom one or more corresponding optimized sequences having an improved function over the target sequence, each optimized sequence having one or more mutations with respect to the target sequence, wherein the machine learning model has been trained on a set of training data comprising native or engineered protein or nucleic acid sequences and additionally, at least a subset of the sequences comprising one or more masked portions and/or at least a subset of the sequences comprising one or mutations introduced therein; and generate output data relating to the one or more optimized sequences.

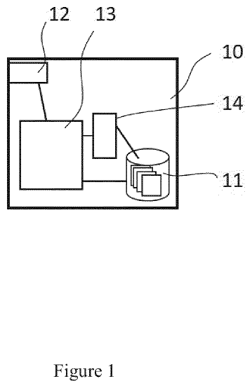

A data processing apparatus can comprise a computing apparatus configured to implement at least some of the herein described features. To provide this the data processing apparatus comprises at least one processor, at least one memory and other internal circuitry and components necessary to perform the tasks. FIG. 1 shows an example of a data processing apparatus 10 comprising processor(s) 12, 13 and memory or memories 11. FIG. 1 further shows connections between the elements of the apparatus and an interface 14 for connecting the data processing apparatus to a data communications system and enabling data communications with the device 10. In some examples, the interface 14 is configured to receive the input target sequence, supply the input target sequence to the machine learning model, and/or present the one or more optimized sequences to a user.

The at least one memory may comprise at least one ROM and/or at least one RAM. The apparatus may comprise other possible components for use in software and hardware aided execution of tasks it is designed to perform. The at least one processor can be coupled to the at least one memory. The at least one processor may be configured to execute an appropriate software code to implement one or more of the following aspects. The software code may be stored in the at least one memory.

### Training of the Machine Learning Model

The apparatus is configured to operate a trained machine learning model which receives an input target protein or nucleic acid sequence, and to generate therefrom an optimized protein or nucleic acid sequence.

Machine learning relates to methods and circuitry that can learn from data and make predictions based on data. In contrast to methods or circuitry that follow static program instructions, machine learning methods and circuitry can include deriving a model from example inputs (such as a set of training data) and then making data-driven predictions.

Machine learning tasks can be categorised into unsupervised learning, supervised learning, and reinforcement learning. In supervised learning, the training data comprises example inputs and their desired outputs. Through iterative optimization of an objective function, supervised learning algorithms learn a function that can be used to predict the output associated with the new inputs. In unsupervised learning, no labels are given to the learning algorithms. The algorithms find patterns or commonalities in the input data and react based on the presence or absence of such patterns or commonalities in each new piece of data. As no labelling of data is required, unsupervised learning can exploit far larger amounts of data. In reinforcement learning, algorithms interact with a dynamic environment in which it must perform a certain goal (such as driving a vehicle).

In an example of the invention, a transformer model is trained on native or engineered protein or nucleic acid sequences (the training data) using unsupervised learning algorithms. Training may use a random initialisation of weights and subsequent optimisation. The term “native” employed herein means natural or existing in nature. The term “engineered” employed herein means artificially synthesised or artificially modified sequences which are functional or working. By “functional” or “working” it is meant that they can perform their intended function, preferably to at least the same extent as any existing native counterparts. In some examples, an engineered sequence may be synthesised de novo. Optionally, an engineered sequence may have at least one improved function or property relative to any existing native counterparts. Thus, by way of example, an intended function of an enzyme could be conversion of substrate to product, and an improved function or property may relate to an improved specificity. Training may be conducted using a combination of native and engineered protein and/or nucleic acid sequences.

The training data may comprise about 5 million, 10 million, 20 million, 30 million, 40 million or 50 million or more native and/or engineered sequences. Increasing the data set to about 100 million, about 200 million, or even larger numbers of sequences may enhance the learned representation. Native sequences may be obtained from one or more known databases including BFD™, Uniref™, UniParc™, Swiss-Prot™, EMBL™, and GenBank™ Other databases include Brenda™, BioCyc™, Mgnify™, MetaEuk™, SMAG™, TOPAZ™, MGV™, GPD™, MetaClust2™, PDB™, SeqRes™, JGI™, NCBI™, and OM-RGC™. In some examples, the training data may comprise less than 5 million native and/or engineered sequences, for example, about 1,000 or about 5,000 or about 10,000, or about 20,000, or about 50,000, or about 100,000, or about 500,000 or about 1 million native and/or engineered sequences.

In addition to the sequences themselves, the training data may comprise distances between amino acid residues within structural representations of the native or engineered protein sequences. In some examples, the training data may comprise distances between at least some or all possible pairs of Ca atoms. In other examples, the training data may comprise configurations relating to the peptide backbone itself or amino acid side chain atoms (for example, CB atoms), or other approximations (for example, centroids). The training data may further comprise structural information pertaining to the sequences which can be obtained from known databases such as PDB™, MODDB™, SWISS-MODEL™, Protein Model Portal™, and AlphaFold™ Protein Structure Database (EMBL-EBI) to improve the model's performance. Structural information may further be derived from predicted protein structures obtained using tools such as TrRosetta™ and AlphaFold™.

Training a machine learning model requires tuning its parameters in order to maximize predictive accuracy. The key test for a machine learning model is the ability to accurately predict labels for inputs it has not seen during training. Therefore, when training the model, it is necessary to estimate the model's performance on data not in the training set. Thus, in particular examples, a portion of the training data (the test set) is removed for model evaluation. Typically, the test set comprises approximately 10 to 20% of the original training data.

The machine learning model is typically a deep learning model comprising a neural network based on deep learning algorithms that use multiple layers to progressively extract higher-level features from the raw input data. Neural networks are generally comprised of various permutations of several different architectures, including, but not limited, to: Convolution Neural Networks (CNNs), Recurrent Neural Networks (RNNs), or Graph Neural Networks (GNNs). In a particular example, the model processes the input sequences through a series of attention blocks combined with convolution layers to capture complex representations that incorporate context from across the entire length of the sequences whilst optionally constructing pairwise interactions (for example, distance between Ca atoms) between all positions in the native protein sequences. Other representations that may be captured by a deep learning model include structural information and configurations relating to the peptide backbone itself or amino acid side chain atoms (for example, CB atoms), or other approximations (for example, centroids), as described above.

In the training process, and with specific regard to native or engineered protein sequences, the model may learn to encode various properties in its representations (i.e. the features derived from the raw native sequences) including biochemical properties, orthologous variations, structural homology, and secondary and tertiary structural information.

In some examples where the native or engineered sequences comprise protein sequences, the model may be trained using a masked modelling language objective. In these examples, each input native sequence is corrupted by replacing a portion of the amino acids with a special mask token. The model is caused to predict the missing tokens from the corrupted sequence by the underlying algorithm. In order to make a prediction for the masked position, the model must identify dependencies between the masked site and the unmasked parts of the sequence. In other examples, where the sequences are aligned, training may comprise the masking of gap tokens and allowing the model to predict deletions in the relevant positions. Analogous objectives may be used for training data comprising nucleic acid sequences.

Other modelling objectives are envisaged. For example, in some examples, the native or engineered sequences may additionally or alternatively be corrupted by one or more mutations which may include substitution mutations, insertion mutations, and/or deletion mutations, and the model may be caused to predict or restore the original residues by the underlying algorithm in training.

Machine learning requires optimization of its trainable weights and accordingly, its predictions, in order to become a trained learning model. This can be achieved by minimizing a loss function. A loss function describes the disparity between the predictions of the model being trained and training data. If predictions deviate too much from the training data, the loss function is high.

Appropriate parameters may be used for the training. In accordance with an example in the context of the present invention, the loss function may be based on three parameters: i) the prediction of whether or not an amino acid or base has been modified at each position of the native protein or nucleic acid sequence; ii) the prediction of the original amino acid at a given position, and iii) specifically for native or engineered protein sequences, the prediction of the relative distance between the Ca atom at any given position, and all other Ca atoms.

If the model's predictions are wrong with respect to any of these parameters, it will be penalized, and the loss function will increase. An aim of training is to improve the accuracy of the model's predictions by minimizing the loss function. Specifically, when the difference in loss between training iterations with respect to the training data becomes smaller than a set value, the machine learning model is considered to be effective at giving predictions for the training data. It may then become necessary to test the model on the remaining test data set aside (see above), using the same loss function. When the loss function with respect to the test data becomes smaller than a set value, the model may be considered trained.

The native or engineered protein or nucleic acid sequences of the training data may be clustered based on sequence identity to balance the training data set and improve performance of the machine learning model. Specifically, clustering prevents the occurrence of any disproportionately-sized dominating clusters containing vast numbers of sequences. The presence of such disproportionately large clusters may skew the learning of the machine learning model towards such clusters and away from the remaining rare/unlikely sequences which the machine learning model may treat as anomalies. The native protein or nucleic acid sequences may be clustered at greater than about 10%, or about 20% or about 30% or about 40%, or about 50%, or about 60%, or about 70%, or about 80%, or about 90%, sequence identity. In some examples, the native protein or nucleic acid sequences may be clustered at about 20% to about 100% sequence identity. This means that every sequence in a given cluster has an identity above the selected threshold. Clustering at 100% sequence identity would therefore mean that all unique sequences in the dataset are used for training the model. Known clustering algorithms may be used for the clustering including CD-HIT and UCLUST.

In some examples, the native or engineered protein or nucleic acid sequences may be filtered to reduce the number of sequences in the training data and/or to improve learning. The sequences may be filtered based on one or more of numerous criteria including but not limited to: genotype, phenotype, structure and sequence identity.

In further examples, training of the machine learning model may comprise causing the machine learning model to output original DNA sequences from input native or engineered protein sequences and vice versa. Conversion and other reverse translation models may be used in such types of training. In these examples, masked modelling objectives may be used and/or mutations may be introduced as described above to enable the machine learning model to be trained to predict the original input sequence. In these examples, the learned representations of the machine learning model may include information relating to the degeneracy of the genetic code, the relationship between codons and amino acids, and the presence of termination codons and start codons.

In preferred examples, the machine learning model may undergo a “fine tuning” step based on the target sequence. Fining tuning comprises further training the machine learning model using a set of native sequences that are homologous to the target sequence. In some examples, the native sequences used in fine tuning have greater than about 10%, greater than about 20%, greater than about 30%, greater than about 35%, greater than about 40%, greater than about 50%, greater than about 60%, greater than about 70%, greater than about 80% or greater than about 90% sequence identity with the target sequence. The fine tuning may be performed using the methods described above for the training of the machine learning model. However, in the fine-tuning process, and in contrast to the training process described above, weights are not initialised randomly but are initialised from the trained model. This enables the model to become trained to perform with greater accuracy. The loss function employed in the fine tuning step may also exclude distance between residues or specifically, Ca pairs, as the variations in distance within homologous sequences are generally not significant.

Native sequences that are homologous to the target sequence may be identified using standard sequence search tools such as BLAST™, HHblits™ and Jackhmmer™, MMSeqs™ and USEARCH™

### Evaluation of the Target Sequence

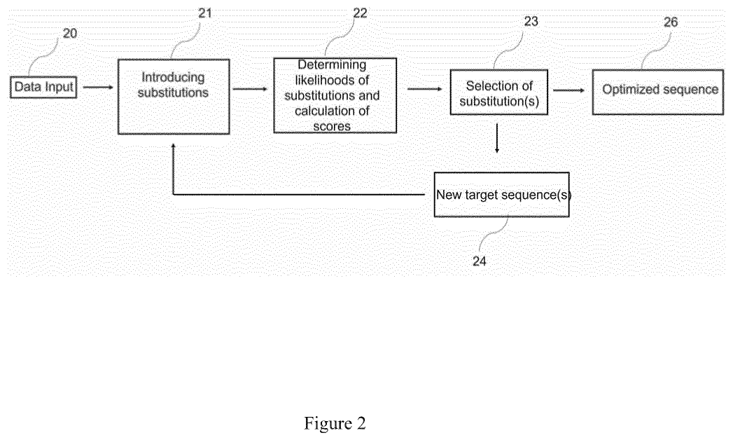

A trained machine learning model can be used for evaluation of a target sequence. FIG. 2 illustrates an example of events once a target sequence is obtained. The data corresponding to the target sequence is inputted (20). After the target sequence has been received and inputted, the target sequence is evaluated for substitutions or mutations (21). If the target sequence comprises a protein sequence, the likelihood of one or more substitutions with each proteinogenic amino acid within a pre-defined set of proteinogenic amino acids at one or more positions within the target sequence is determined. If the target sequence comprises a nucleic acid sequence, the likelihood of one or more substitutions with each base in a pre-defined set of bases at one or more positions within the target sequence is determined. In preferred examples, the likelihood of substitutions with a pre-defined set of amino acids or pre-defined set of bases at two or more positions of the target sequence are determined. In these examples, the likelihood is based on a combination of substitutions at the two or more positions. The likelihood represents the probability of the given amino acid or base occurring at their respective positions, or preferably, the probability of a given combination of amino acids or bases occurring at their respective positions, based on parameters identified and evaluated by the trained machine learning model. In determining the likelihood of each substitution, the model preferably assesses the interaction between amino acids or bases at each possible position simultaneously.

In some examples, every position of the target sequence is evaluated for likelihood of substitutions or mutations with the pre-defined set of proteinogenic amino acids or pre-defined set of bases. In other examples, a set of positions of the target sequence, where the set does not comprise every position of the target sequence, is evaluated for likelihood of substitutions or mutations with the pre-defined set of proteinogenic amino acids or pre-defined set of bases. In some examples, the target sequence may comprise a protein sequence which is converted to a corresponding nucleic acid sequence for evaluation and optimization. In other examples, the target sequence may comprise a nucleic sequence which is converted to a corresponding protein sequence for evaluation and optimization. Standard conversion tools and reverse translation tools may be used in such examples.

Proteinogenic amino acids are canonical amino acids that are incorporated biosynthetically into proteins during translation. There are twenty proteinogenic amino acids in the standard genetic code. These are: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. An additional two may be incorporated by special translation mechanisms. These are: selenocysteine and pyrrolysine. In some examples, the pre-defined set of proteinogenic amino acids may comprise or consist of those proteinogenic amino acids in the standard genetic code. In other examples, the pre-defined set of proteinogenic amino acids may additionally comprise or consist of selenocysteine and pyrrolysine. In further examples, the pre-defined set of proteinogenic amino acids is a subset of the proteinogenic amino acids defined above.

In other examples, amino acids of a target sequence may be substituted with modified (non-proteinogenic) amino acids, and scores based on the likelihood of substitutions calculated as above. Examples of modified or non-proteinogenic amino acids include but are not limited to selenocysteine, pyrrolysine, hydroxylysine, desmosine, ornithine, norleucine, sarcosine and others. In these examples, the machine learning model may have been trained accordingly using protein sequences comprising modified amino acids.

The pre-defined set of bases may comprise or consist of a set of standard bases. Standard bases are the canonical nitrogenous bases adenine, thymine, uracil, cytosine and guanine. The predefined set of bases may comprise or consist of all the above bases, or a subset thereof. In other examples, bases of a target nucleic acid sequence may be substituted with modified (non-canonical) bases, and scores for the likelihood of substitutions calculated as above. Examples of modified or non-canonical bases include but are not limited to methylcytosine, hydroxymethylcytosine, formylcytosine, carboxycytosine, dihydrouracil, pseudouracil, 4-thiouracil, 1-methylguanine, 7-methyladenine and others. In these examples, the machine learning model may have been trained accordingly using nucleic acid sequences comprising modified bases.

The trained machine learning model, as determined by its algorithms, calculates a score for the likelihoods of the substitutions based on a scoring function (22).

In some examples, the scoring function is equal to the likelihood itself (e.g. f(likelihood)=likelihood×1). In other examples, the function is based on likelihood but includes another input such as a threshold. In these examples, the function would return a score of 1 or 0 only (e.g. f(likelihood, threshold={1, likelihood>threshold; 0, likelihood<threshold}). The function may include further inputs. For example, the function may require one or more positions to be fixed during optimization. This would be accounted for when calculating the scores such that substitutions at such fixed positions would score less favourably. In other examples, and optionally in the absence of any input function requiring one or more positions to be fixed, the wild-type amino acid or base may be included in the pre-defined set of amino acids or pre-defined set of bases that is used for substitution at the one or more positions within the target sequence. In these examples, substitutions with the wild-type amino acid or base may be selected over other amino acids or bases in the pre-defined set based on calculated scores.

One or more substitutions may be selected based on the calculated scores to generate one or more new mutated target sequences, each comprising one or more substitutions (23,24). In an example of the invention, one or more substitutions with a calculated score according to a pre-defined scoring function are selected to generate one or more new mutated target sequences. There are various possibilities of how to select substitutions based on calculated scores, and the method of selection depends on the application. In some examples where the scoring function is equal to likelihood, the higher the score (or the lower the score in a reverse scoring system), the greater the likelihood of the amino acid or base at their respective positions. In these examples, one or more substitutions having a calculated score corresponding to the greatest likelihood may be selected. In other examples, at least two or more substitutions, respectively, each having a greater likelihood than their wild-type counterparts, may be considered favourable, and accordingly selected.

In some instances, single-point mutations which are scored favourably may not be effective in combination with other favourably scored single-point mutations (i.e. the effects of each individual mutation may not be additive). Accordingly, in examples as described above, the likelihoods of substitutions at two or more respective positions of the target sequence may be determined. In these examples, the machine learning model is able to produce likelihoods for combinations of substitutions. In other words, the likelihood of a substitution at a given position may be determined based on its combination with one or more other substitutions or mutations in the target sequence, rather than as a single-point or isolated substitution or mutation. In these examples, even though a likelihood may be based on a combination of substitutions, the machine learning model may nevertheless be configured select only a single substitution from the combination.

The present invention may obviate the need to perform evaluation of the large numbers of possible combinations of substitutions or mutations that could be provided in any given target sequence. By simultaneously evaluating the likelihood of substitutions or mutations in each combination, the time taken to arrive at an optimized sequence may be significantly reduced, and the optimization process may be enhanced.

In some examples, only the most favourably scoring substitution(s) of the target sequence are selected to generate the new mutated target sequence(s). In some exemplary scoring systems, the most favourably scoring substitutions may be those with the highest scores. In other examples, all substitutions having a favourable score are selected to generate the new mutated target sequence. In further examples, a randomly selected subset of those with the most favourable calculated scores or with at least a favourable score, are selected to generate the one or more new mutated target sequences. For example, a threshold score may be determined. One or more scoring more favourably than the threshold may be randomly selected to generate the new mutated target sequence. Alternatively, all substitutions scoring more favourably than the threshold may be selected to generate the new mutated target sequence. In some examples, the substitutions are ranked in order of their score, and a pre-selected number of the favourably scoring substitutions are selected randomly to generate one or more new mutated target sequences. In yet further examples, a non-randomly selected subset of substitutions from those with the most favourable score or with at least a favourable score, are selected to generate the one or more new mutated target sequences.

In some examples, substitutions or mutations may further be introduced randomly into the target sequence (either the original target sequence or a new target sequence comprising one or more substitutions obtained during the iterative optimization process at any or each stage of the iterative process (for example, before or during the optimization process) to increase diversity, and subsequently evaluated as above. In other examples, substitutions or mutations may be introduced on the basis of a position-specific scoring matrix (PSSM). A PSSM is assembled from an alignment of the target sequence with homologous sequences. The matrix enables an assessment of which amino acids (or bases) exist at each position of the sequence and their frequency or likelihood of observation. Substitutions or mutations may accordingly be selected and introduced into the target sequence as defined above based on probability derived from PSSM. In yet further examples, one or more positions of the target sequence as defined above may be masked at any or each stage of the optimization process and the likelihood of each amino acid or base at the masked position determined.

In preferred examples, the processes described above of evaluating and assessing the likelihood of substitutions at one or more positions of the sequence, calculating scores, and selecting one or more substitutions, are repeated on the newly generated target sequences to generate further new target sequences (25), until no further substitutions are yielded by the system, and one or more optimized sequences (26) are obtained. In other words, over multiple iterations of the processes, the neural network converges to a solution. In other examples, the processes described above of evaluating and assessing the likelihood of substitutions at one or more positions of the sequence, calculating scores, and selecting one or more substitutions, are repeated until the apparatus has been running for a selected amount of time. Output sequences may also be collected after each iterative step for further analysis, thus obtaining all variants.

The number of substitutions in the optimized sequences obtained by the method and using the apparatus of the present invention is not particularly limited. The one or more optimized sequences may have, for example, from 1 to 500 substitutions, or from 1 to 200 substitutions, or from 50 to 200 substitutions, or from 100 to 200 substitutions, or from 50 to 100 substitutions, relative to the original target sequence.

### Characteristics of Optimized Sequence

The target sequence may be any protein or nucleic acid sequence. In preferred examples, the target sequence comprises an enzyme sequence which is optimized by the invention. In these examples, the one or more improved functions of the optimized sequence includes, but is not limited to a function selected from: k_{cat}/Kₘ, k_{cat}, Kₘ, thermostability, pH stability, ionic strength stability, solvent stability, resistance to one or more inhibitors, resistance to a chaotropic agent, resistance to an ionic detergent, shelf-life, expressibility in recombinant systems, and adsorption to a plastic.

k_{cat} represents the rate of reaction at saturating substrate concentration and is the maximal number of molecules of substrate converted to product per active site per unit time when the enzyme is saturated with substrate. Accordingly, in some examples, optimization may include increasing k_{cat} relative to the target sequence.

Enzymes have varying affinities towards their substrates. The Kₘ (Michaelis constant) of an enzyme represents the substrate concentration at which half the enzyme's active sites are occupied by substrate. A high Kₘ signifies a low affinity of the enzyme for a particular substrate (and that a relatively large amount of substrate is needed to saturate the enzyme active sites). Conversely, a low Kₘ signifies a high affinity of the enzyme for a particular substrate (and that a relatively small amount of substrate is needed to saturate the enzyme active sites). Accordingly, in some examples, optimization may include lowering the Kₘ relative to the target sequence.

Some enzymes catalyse the conversion of different substrates to different products. In these cases, the k_{cat}/Kₘ value, or specificity constant, of the various substrates can be compared. That substrate with the highest value is the “best” substrate for the enzyme (i.e. the more specific the enzyme is for that substrate). Accordingly, in some examples, optimization may include an increase in specificity towards a given substrate, as reflected by an increase in k_{cat}/Kₘ of the optimized sequence as compared to the enzyme encoded by the target sequence, for a given substrate.

The rate of any reaction is limited by the rate at which reactant molecules collide. The diffusional limiting rate for a bimolecular reaction is 108 to 109 M⁻¹ s⁻¹. Enzymes that exhibit ratios of k_{cat}/Kₘ near 108 to 109 M⁻¹ s⁻¹ (close to the maximum allowed by the rate of diffusion) have achieved catalytic perfection. Accordingly, in some examples, optimization may comprise a change in the k_{cat}/Kₘ value of the enzyme towards the diffusional limiting rate, reflecting improved catalytic efficiency.

Thermostability relates to the effect of temperature on the activity of enzymes. Typically, enzyme activity increases with temperature up to an optimum temperature. As the temperature is increased beyond the optimum temperature, enzyme activity diminishes due to the disruption of bonds, particularly hydrogen bonds, which maintain the secondary and tertiary structures of enzymes. Once the tertiary structure of the enzyme is lost, the enzyme is considered denatured and essentially, inactive. The temperature at which this occurs is the denaturation temperature. Optimization may include improving thermostability of the target sequence and more specifically, increasing the denaturation temperature such that the optimized enzyme retains activity (i.e. is stable) at a wider range of temperatures or exhibit improved activity at a desired temperature. Optimization may further include increasing the melting temperature of the protein wherein the melting temperature may be defined as the temperature at which the free energy change of unfolding (ΔG) is zero and 50% of the population of protein molecules is in the folded state whilst 50% of the population of protein molecules is in the unfolded state.

pH stability relates to the effect of pH on the activity of enzymes. Enzymes are amphoteric molecules containing a large number of acid and basic groups, mainly situated on their surface. The charges on these groups will vary, according to their acid dissociation constants, and with the pH of their environment. This will, in turn, affect the total net charge of the enzymes and the distribution of charge on their exterior surfaces, in addition to the reactivity of the catalytically active groups. Taken together, the changes in charges with pH affect the activity, structural stability and solubility of the enzyme.

Typically, enzymes are most active at an optimal pH. As the pH is decreased or increased beyond the optimal the pH, enzyme activity decreases due to denaturation of the enzyme. Accordingly, optimization may include improving the pH stability of the target sequence such that the optimized enzyme retains activity at a wider range of pH values and/or exhibits improved activity at a desired pH.

Ionic strength of a medium in which an enzyme is provided is an important parameter affecting enzyme activity. This is especially relevant where catalysis depends on the movement of charged molecules relative to each other. Thus, both the binding of charged substrates to enzymes and the movement of charged groups within the catalytic active site will be influenced by the ionic composition of the medium. Accordingly, optimization may include improving ionic strength stability such that the optimized enzyme has improved activity at a given ionic strength or range of ionic strengths or retains activity over a wider range of ionic strengths.

Solvent stability relates to the effect of different solvents on enzyme activity. Optimization may include improving solvent stability such that the optimized enzyme has improved activity at in a given solvent or group of solvents, or retains activity over a wider range of solvents.

Enzymes may be susceptible to inhibition by various specific and non-specific inhibitors, chaotropic agents and ionic detergents. Optimization may include improved resistance to, and thus improved activity in the presence of, one or more of specific and non-specific inhibitors, chaotropic agents and ionic detergents, relative to the enzyme encoded by the original target sequence.

The shelf-life of enzymes relates to their storage stability. Optimization may include improved retention of activity over longer periods of time and/or over a wider range of temperatures or at a desired temperature.

Many factors influence the expression of recombinant proteins including aggregation, degradation, toxicity to the host cell, and stability. Accordingly, optimization may include an increase in expressibility of the target sequence which may be an enzyme sequence, as determined by any one of the above parameters or any other parameter.

Optimization may further include improving the processivity of enzymes such as DNA or RNA polymerase or reverse transcriptase. In other examples, optimization may include improving the error rate or fidelity for enzymes such as DNA or RNA polymerase or reverse transcriptase.

Proteins often bind in a non-specific manner to solid surfaces such as plastic via hydrophobic and electrostatic interactions. In many circumstances, this is undesirable. For example, during research and development, proteins bind to different lab consumables such as microplates, storage tubes, pipette tips, and centrifuge tubes. During commercialization, proteins may bind to the primary container (glass or plastic vial) storing them. The performance of biomedical devices, biosensors, and biomicrofluidic systems may also be affected by protein adsorption. In other circumstances, protein adsorption to a plastic surface is required for the manufacture of solid-phase separation systems or solid-phase assays. Accordingly, optimization may include reducing or modifying adsorption capacity of the target sequence to a solid surface such as plastic.

The present invention is advantageous in that the process of generating one or more optimized sequences from the target sequence using the trained machine learning model may not rely on the introduction of random mutations and subsequent evaluation of the randomly mutated sequences to identify optimized sequences. Rather, in the present invention, substitutions may be proposed and evaluated by the trained model, greatly reducing the search time for optimized sequences. Additionally, as the model may be able to produce likelihoods for combinations of substitutions in a single step, convergence time to an optimized sequence and the size of the sequence space to be sampled, are further reduced. The invention may provide a high success rate for obtaining optimized sequences.

The examples described herein may be implemented by computer software stored in a memory and executable by at least one data processor or by hardware, or by a combination of software and hardware. Further in this regard it should be noted that any of the above procedures may represent program steps, or interconnected logic circuits, blocks and functions, or a combination of program steps and logic circuits, blocks and functions. The software may be stored on such physical media as memory chips, or memory blocks implemented within the processor, magnetic media such as hard disk or floppy disks, and optical media such as for example DVD and the data variants thereof, CD. The memory may be of any type suitable to the local technical environment and may be implemented using any suitable data storage technology, such as semiconductor-based memory devices, magnetic memory devices and systems, optical memory devices and systems, fixed memory and removable memory. The data processors may be of any type suitable to the local technical environment, and may include one or more of general purpose computers, special purpose computers, microprocessors, digital signal processors (DSPs), application specific integrated circuits (ASIC), gate level circuits and processors based on multi core processor architecture, as non-limiting examples.

While certain aspects of the invention may be illustrated and described as block diagrams, flow charts, or using some other schematic pictorial representation, it is well understood that these blocks, apparatus, systems, techniques and methods described herein may be implemented at least in part in, as non-limiting examples, hardware, software, firmware, special purpose circuits or logic, general purpose hardware or controller or other computing devices, or some combination thereof.

The following are intended as examples only and do not limit the present disclosure.

### EXAMPLES

### Example 1—Single Mutation without Mask Optimization of Beta-Glucosidase

Sequence optimization for beta-glucosidase was performed using a target wild-type protein sequence and inputting it into an algorithm. After receiving the likelihoods for each position and each amino acid per position from the algorithm, a single mutation with the highest likelihood over all positions was picked using the Argmax™ selection function. The resulting sequence with one mutation was used as the new input to repeat the process until convergence. From the sequences collected after each iteration, four variants were picked at 70-90% identity range compared to the wild-type sequence, including the final converged, optimized sequence. A His-tag and linker were appended to the C-terminal end of each optimized beta-glucosidase protein sequence, reverse translated, and codon optimized for expression in E. coli.

Synthesized (Twist Bioscience) optimized beta-glucosidase gene fragments (OP5-OP8) were cloned into pET-21 plasmids using In-Fusion® HD Cloning Kit (Takara Bio) and sequenced after amplification in K12 based E. coli strain. Purified plasmids carrying optimized genes were transformed into E. coli using standard heat-shock (CaCl2 competent E. coli shocked with 42° C. for 30 sec) or electroporation (electro-competent E. coli subjected to 18 000 V/cm) protocol. Expression was carried out in media consisting of 2% tryptone (Formedium), 1% yeast extract (Formedium), 2% NaCl (Roth) and 100 μg/mL ampicillin (Sigma-Aldrich) using T7 RNA polymerase/promoter system. The cells were grown at 37° C., induced with 0.5 mM IPTG (Sigma-Aldrich) and 0.1% L-rhamnose (Roth) at OD600=0.8 and grown for 22 h at 16° C. afterwards. The proteins were expressed with 6 C-terminal histidine residues to facilitate purification.

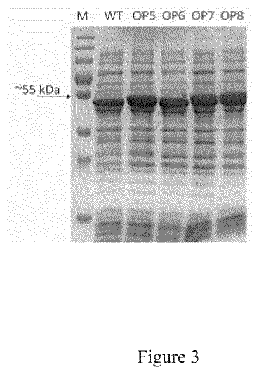

SDS-PAGE analysis of cell lysates was performed to verify expression of the variants. FIG. 3 illustrates that the WT sequence and each of the 4 optimised variants (OP5-OP8) were expressed successfully (M=molecular weight marker).

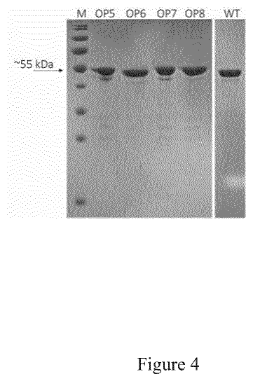

Recombinant proteins were purified by immobilized metal affinity chromatography using Ni Sepharose High Performance nickel-charged IMAC resin (Cytiva). The cells were lysed in 50 mM Tris-HCl, pH 8.0, 300 mM NaCl, 10 mM imidazole (Sigma-Aldrich) by sonication and clear lysate loaded onto the resin. The resin was then washed with buffer containing 25-50 mM imidazole and proteins eluted with buffer containing 500 mM imidazole. Imidazole was removed by dialysis. Protein concentrations measured using NanoDrop™ 8000 Spectrophotometer (Thermo Fisher Scientific), molecular weight and extinction coefficient was taken into account for each protein individually. SDS-PAGE analysis of purified protein preparations is illustrated in FIG. 4. It can be seen that variants OP5-OP8 were purified effectively.

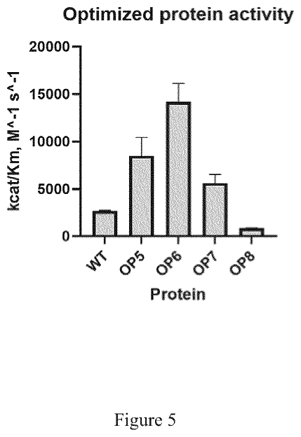

Protein activity was measured using para-Nitrophenyl β-D-glucopyranoside (pNPG, Sigma-Aldrich) as a substrate. During the reaction pNPG is broken down and para-nitrophenol is released producing a change in absorbance. The OP variant sequences were mixed with 1 mM pNPG in 50 mM Hepes, pH 7.5, 150 mM NaCl, 0.5 mg/ml BSA and absorbance measured every 41 sec at 405 nm for 1 h. Final activity data was obtained by analyzing the resulting kinetic curves and calculating kcat/Km values. These activity values were compared to purified WT beta-glucosidase and the results are illustrated in FIG. 5. Optimized proteins are in most cases more active than the WT sequence—activity values are up to 5.3 times higher than that of wild-type.

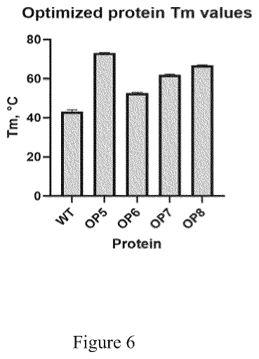

Protein melting temperature was determined by performing a thermal shift assay. GloMelt (Biotium) fluorescent dye was used to detect protein unfolding and measure thermal stability according to manufacturer's recommendations. Briefly, each protein was mixed with 1×GloMelt in 50 mM Hepes, pH 7.5; 150 mM NaCl and melting curve was recorded using the following program on CFX Touch Real-Time PCR Detection System (Bio-Rad): Melt curve 20° C. to 95° C., increment 0.05° C. for 30 s+plate read. Obtained Tm values were compared to purified wild type beta-glucosidase. FIG. 6 illustrates that optimized proteins OP5-OP8 have melting temperatures that are higher than wild-type-Tm values are higher by up to 30° C. as compared to wild-type.

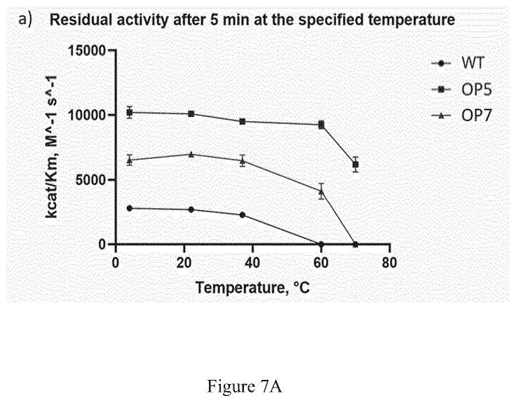

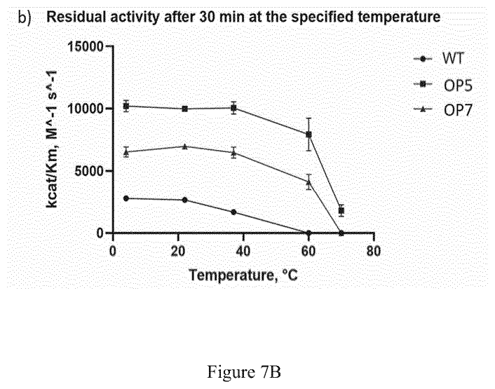

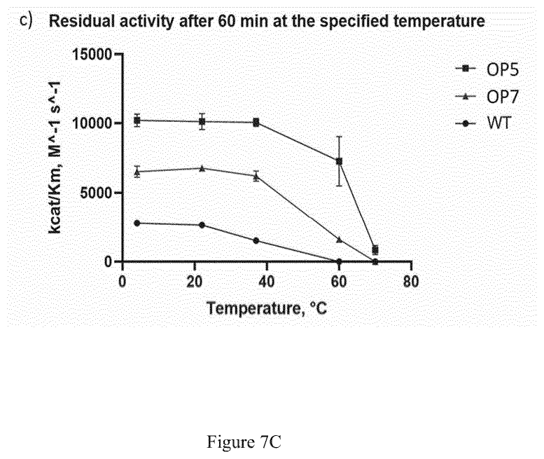

Thermal stability of the most active and stable variants (OP5 and OP7) was also confirmed by evaluating protein residual activity. Residual activity was measured using pNPG as a substrate. The proteins were heated for 5, 30 or 60 minutes at elevated temperatures before mixing with 1 mM pNPG in 50 mM Hepes, pH 7.5, 150 mM NaCl, 0.5 mg/ml BSA buffer and recording kinetic curves. Kinetic curves were obtained by measuring absorbance every 41 sec at 405 nm for 1 h. The resulting curves were analyzed, kcat/Km values calculated. These residual activity values were compared to purified WT beta-glucosidase. FIGS. 7A, 7B and 7C illustrate that OP5 and OP7 were able to endure temperatures up to 70° C., even after 60 minutes of heating, and remain active (albeit with some loss in activity). The OP5 variant remained ˜2.6 times more active than non-heated wild-type after incubation at 60° C. for 1 hour.

### Example 2—Multiple Mutations without Mask Optimization of Beta-Glucosidase

Sequence optimization for beta-glucosidase was performed starting from a wild-type protein sequence and inputting it into an algorithm. After receiving the likelihoods for each position and each amino acid per position from the algorithm, at each position, a single mutation with highest likelihood was picked using the Argmax™ selection function. The resulting sequence with multiple mutations was used as the new input to repeat the process until convergence. From the sequences collected after each iteration, 3 sequences were picked at random (OP9-OP11). A His-tag and linker were appended to the C-terminal end of each beta-glucosidase protein sequence, reverse translated, and codon optimized for expression in E. coli.

The optimised variants were recombinantly expressed and purified by the methods described in Example 1 (data not shown).

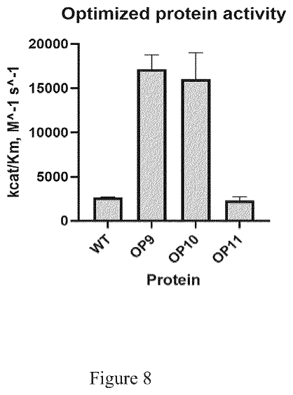

Protein activity of each of the optimized variants were measured by the methods described in Example 1, and compared to purified wild-type beta-glucosidase. The results are illustrated in FIG. 8. It can be seen that optimized proteins are more active than the wild-type-activity values are up to 6.5 times higher than that of wild-type.

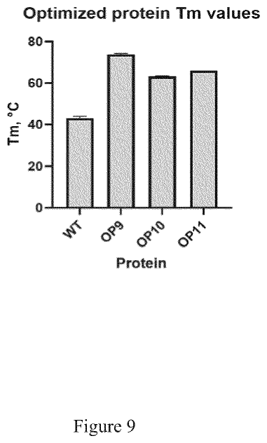

Protein melting temperature was determined by performing thermal shift assay, as described in Example 1. Obtained Tm values were compared to purified wild type beta-glucosidase. The results are illustrated in FIG. 9. Optimized proteins 9-11 have melting temperatures that are higher than wild-type-Tm values are higher by up to 30.7° C. as compared to wild-type.

### Example 3—Single Mutation with Masked Optimization of Beta-Glucosidase

Sequence optimization for beta-glucosidase was performed starting from the wild-type protein sequence with one position randomly masked and inputting it into the algorithm. After receiving the likelihoods for each position and each amino acid per position from the algorithm, one single mutation with highest likelihood per masked position was picked using the Argmax™ selection function. The resulting sequence with one mutation was again masked at one random position and used as the new input to repeat the process for 700 iterations. Afterwards, the process was repeated with the collected sequences without masking where one mutation with highest likelihood was picked over all positions using the Argmax™ selection function, and the process repeated until convergence. From the resulting sequences, 7 sequences were picked at 65-95% identity range compared to the WT sequence (OP12-OP18). A His-tag and linker were appended to the C-terminal end of each beta-glucosidase protein sequence, reverse translated into ge and codon optimized for expression in E. coli.

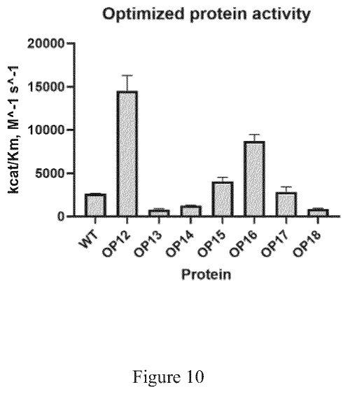

Protein activity of each of the optimized variants were measured by the methods described in Example 1, and compared to purified wild-type beta-glucosidase. The results are illustrated in FIG. 10. It can be seen that optimized proteins are more active than the wild-type-activity values are up to 5.5 times higher than that of wild-type.

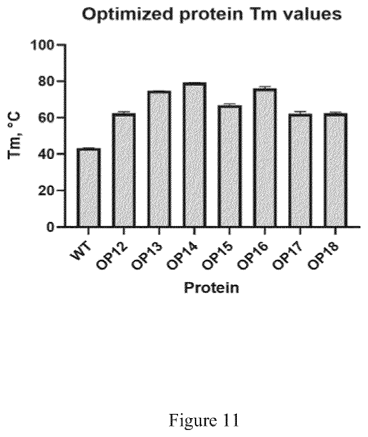

Protein melting temperature was determined by performing thermal shift assay, as described in Example 1. Obtained Tm values were compared to purified wild type beta-glucosidase. The results are illustrated in FIG. 11. Optimized proteins 12-18 have melting temperatures that are higher than wild-type-Tm values are higher by up to 36° C. as compared to wild-type.

### Example 4—Single Mutation without Mask Optimization of Flavin Reductase hypE

Sequence optimization for flavin reductase hpyE was performed starting from the wild-type protein sequence and inputting it into the algorithm. After receiving the likelihoods for each position and each amino acid per position from the algorithm, one single mutation with highest likelihood per all positions was picked using the Argmax™ selection function. The resulting sequence with one mutation was used as the new input to repeat the process until convergence. From the sequences collected after each iteration, 4 variants were picked at 80-97% identity range compared to the wild type sequence, including the final converged sequence (OP1-OP4). A His-tag and linker were appended to the C-terminal end of each hpyE protein sequence, reverse translated into genes and codon optimized for expression in E. coli.

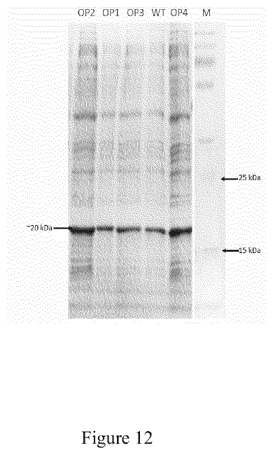

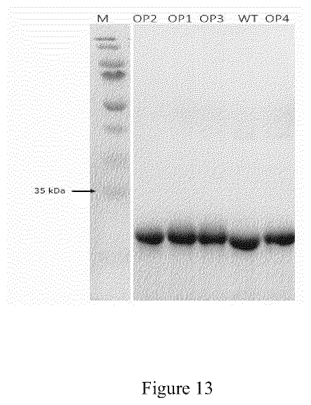

The optimized variants were recombinantly expressed in E. coli and purified by the methods described in Example 1. SDS-PAGE analysis of cell lysates was performed to verify expression of the variants. FIG. 12 illustrates that each of the 4 optimised variants (OP1-OP4)_were expressed successfully (M=molecular weight marker). SDS-PAGE analysis of purified protein preparations is illustrated in FIG. 13. It can be seen that variants OP1 to OP4 were purified effectively.

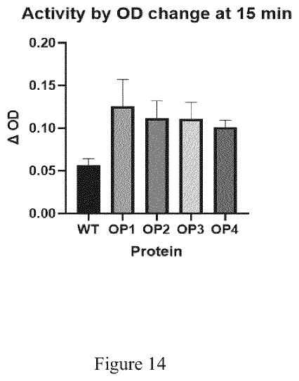

Protein activity of each of the optimized variants was measured using flavin mononucleotide (FMN, Sigma-Aldrich) and nicotinamide adenine dinucleotide (NADH, Carl Roth) as substrates. During the reaction hpyE catalyzes the reduction of FMN to a reduced flavin. The proteins (0.0015 μM final concentration) were mixed with 10 μM FMN and 150 μM NADH in 50 mM Tris-HCl, pH 7.5, 0.3 mg/ml BSA and absorbance measured every 13 sec at 340 nm for 1 h. Final activity data (delta OD) was obtained by subtracting OD values measured at 18 s from OD values measured at 15 min 20 s. These activity values were compared to purified wild-type flavin reductase hpyE. The results are illustrated in FIG. 14. It can be seen that each of the optimized proteins are more active than wild-type enzyme.

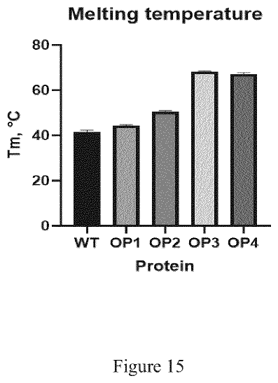

Protein melting temperature was determined by performing a thermal shift assay, as described in Example 1. Obtained Tm values were compared to purified wild-type flavin reductase hypE. The results are illustrated in FIG. 15. Optimized proteins 1-4 have melting temperatures that are higher than wild-type-Tm values are higher by up to 27.2° C. as compared to wild-type.

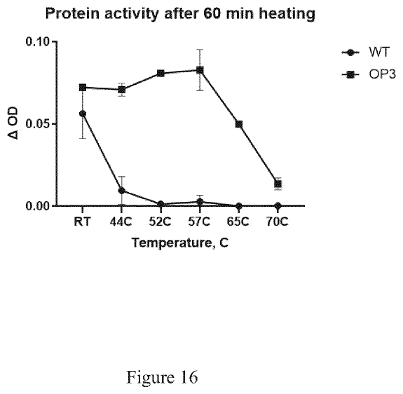

Thermal stability of one of the most active and stable variant OP3 was confirmed by calculating protein residual activity. Residual activity was measured using FMN and NADH as substrate. 3 nM concentration OP3 was heated for 60 minutes at elevated temperatures before mixing with 10 μM FMN and 150 μM NADH in 50 mM Tris-HCl, pH 7.5, 0.3 mg/ml BSA buffer. Final OP3 concentration in the reaction is 1.5 nM. Kinetic curves were obtained by measuring absorbance every 13 sec at 340 nm for 1 h. Residual activity data (delta OD) was obtained by subtracting OD values measured at 18 s from OD values measured at 15 min 28 s. Residual activity values were compared to purified wild-type flavin reductase hpyE. As illustrated in FIG. 16, OP3 was able to endure temperatures up to 65° C. and remain active (with some loss in activity).

The foregoing description provides by way of exemplary and non-limiting examples a full and informative description of exemplary embodiments of the invention. However, various modifications and adaptations may become apparent to those skilled in the relevant arts in view of the foregoing description, when read in conjunction with the accompanying drawings and the appended claims. All such and similar modifications of the teachings of this invention will still fall within the scope of this invention as defined by the claims.

1. An apparatus for generating an optimized protein or nucleic acid sequence from a target protein or nucleic acid sequence, the apparatus comprising at least one processor and at least one memory including a computer program code, the at least one memory and computer program code configured to, with the at least one processor, cause the apparatus to at least:
operate a machine learning model configured to receive an input target protein sequence or a nucleic acid sequence, and to generate therefrom one or more corresponding optimized sequences having an improved function over the target sequence, each optimized sequence having one or more mutations with respect to the target sequence, wherein the machine learning model has been trained on a set of training data comprising native or engineered protein or nucleic acid sequences, and additionally, at least a subset of the sequences comprising one or more masked portions and/or at least a subset of the sequences comprising one or more mutations introduced therein; and
generate output data relating to the one or more optimized sequences.

2. The apparatus of claim 1, wherein the apparatus is configured to perform the following steps after the target sequence has been received:
i) determine likelihoods of substitutions with a pre-defined set of proteinogenic amino acids at one or more positions within the target sequence when the target sequence is a protein sequence, or with a pre-defined set of bases at one or more positions within the target sequence when the target sequence is a nucleic acid sequence;
ii) calculate scores for the likelihoods of the substitutions at the one or more positions based on a scoring function;
iii) select one or more substitutions based on the calculated scores to generate one or more new mutated target sequences each comprising one or more substitutions; and
iv) repeat steps i) to iii) until no further substitutions with an improved score are yielded at each corresponding position; or repeat steps i) to iii) until the apparatus has performed for a selected amount of time.

3. The apparatus of claim 2, wherein the apparatus is configured to determine the likelihoods of substitutions with the pre-defined set of proteinogenic amino acids at two or more respective positions within the target sequence when the target sequence is a protein sequence, or with the pre-defined set of bases at two or more respective positions within the target sequence when the target sequence is a nucleic acid sequence, wherein the likelihoods are based on the combined substitutions at the two or more positions.

4. The apparatus of claim 2, wherein the scoring function is equal to the likelihood.

5. The apparatus of claim 2, wherein the scoring function further comprises an input that is independent of likelihood.

6. The apparatus of claim 2, wherein the apparatus is configured to introduce one or more mutations into the received target sequence and/or into the one or more new target sequences obtained in step iii).

7. The apparatus of claim 6, wherein the introduction of one or more mutations is based on probability derived from a position-specific scoring matrix.

8. The apparatus of claim 1, wherein the input target sequence comprises a protein sequence.

9. The apparatus of claim 8, wherein the protein is an enzyme.

10. The apparatus of claim 8, wherein the one or more optimized sequences exhibit one or more improved functions relative to the target sequence selected from: K_{cat}/Kₘ, K_{cat}, Kₘ, thermostability, pH stability, specificity, ionic strength stability, solvent stability, resistance to one or more inhibitors, resistance to a chaotropic agent, resistance to an ionic detergent, shelf-life, expressibility in recombinant systems, and adsorption to a plastic.

11. (canceled)

12. The apparatus of claim 1, wherein the machine learning model is trained on 20% to 100% cluster representatives of the native sequences.

13. The apparatus of claim 12, wherein the machine learning model is trained on about 50% cluster representatives of the native sequences.

14. The apparatus of claim 1, wherein the machine learning model is trained on distances between amino acid residues within structural representations of the native or engineered protein sequences.

15. The apparatus of claim 1, wherein the machine learning model is constructed from multi-attention blocks combined with convolution layers.

16. The apparatus of claim 1, wherein the machine learning model has been further trained on a set of homologous sequences that have greater than about 35% sequence identity to the target sequence.

17. The apparatus of claim 1, wherein the apparatus further comprises a user interface which is configured to receive the input target sequence and to supply the input target sequence to the machine learning model, and wherein the user interface is configured to present the one or more optimized sequences to a user.

18. (canceled)

19. A computer-implemented method of generating one or more optimized protein sequences or nucleic acid sequences from a target protein sequence or nucleic acid sequence, using a trained machine learning model;
wherein the machine learning model has been trained based on:
a) a set of training data comprising native or engineered protein or nucleic acid sequences; and
b) at least a subset of the native or engineered protein or nucleic acid sequences comprising one or more masked portions and/or at least a subset of the native or engineered protein or nucleic acid sequences comprising one or mutations introduced therein; and
wherein the machine learning model is configured use the training data in order to become a trained machine learning model; and
wherein the method comprises:
i) receiving as input the target sequence;
ii) causing the trained machine learning model to evaluate the inputted target sequence; and
iii) based on the evaluation, generating one or more optimized sequences corresponding to the target sequence, each optimized sequence having an improved function over the target sequence, and one or more mutations with respect to the target sequence; and
iv) outputting data relating to the one or more generated optimized sequences.

20. The method of claim 19, wherein in step (ii), evaluation of the target sequence comprises:
v) determining likelihoods of substitutions with a pre-defined set of proteinogenic amino acids at one or more positions within the target sequence when the target sequence is a protein sequence, or with a pre-defined set of bases at one or more positions within the target sequence when the target sequence is a nucleic acid sequence;
vi) calculating scores for the likelihoods based on a scoring function;
vii) selecting one or more substitutions based on the calculated scores to generate one or more new mutated target sequences each comprising one or more substitutions; and
viii) repeating steps v) to vii) until no further mutations with an improved score are yielded at each corresponding position; or repeating steps v) to vii) until the apparatus has performed for a selected period of time.

21-25. (canceled)

26. The method of claim 19, wherein in step b), the introduced mutations are random.

27-35. (canceled)

36. The method of claim 19, wherein the machine learning model has been trained based on a loss function related to predicting the original amino acid or original base at any given position of the protein sequence or nucleic acid sequence, predicting a mutated amino acid or a mutated base at any given position of the protein sequence or nucleic acid sequence, and if the training data comprises native protein sequences, the distance between Ca atoms of the protein sequences.

37-38. (canceled)
